(19) United States
(12) Patent Application Publication
(10) Pub. No.: US 2025/0320557 A1
(43) Pub. Date: Oct. 16, 2025

(54) USE OF ABCF1 IN METHODS OF DIAGNOSING AND MONITORING INFLAMMATORY AND/OR AUTOIMMUNE DISEASE

(71) Applicant: MYND Life Sciences Inc., Kelowna (CA)

(72) Inventor: Wilfred A. JEFFERIES, Surrey (CA)

(21) Appl. No.: 18/546,939

(22) PCT Filed: Feb. 16, 2022
(86) PCT No.: PCT/CA2022/050220

§ 371 Date: Aug. 17, 2023

Related U.S. Application Data
(60) Provisional application No. 63/150,249, filed on Feb. 17, 2021.
(60) Provisional application No. 63/275,650, filed on Nov. 4, 2021.
(60) Provisional application No. 63/284,723, filed on Dec. 1, 2021.

(51) Int. Cl. C12Q 1/6883; G01N 33/564

(52) U.S. Cl. CPC C12Q 1/6883; G01N 33/564; C12Q 2600/158; G01N 2800/52; G01N 2800/7095

(57) ABSTRACT

The present invention relates to the use of ABCF1 in diagnosing and/or monitoring inflammatory and autoimmune diseases. In certain embodiments, the methods comprise a a) collecting samples at pre-determined intervals from a subject; b) measuring the level of ABCF1 in each of the samples collected from the patient; and c) comparing the level of ABCF1 in each of the samples.

### REFERENCE TO SEQUENCE LISTING SUBMITTED ELECTRONICALLY

The content of the electronically submitted sequence listing (Name: 5345_0010003_SeqListing_ST25.txt; Size: 19,885 bytes; and Date of Creation: Apr. 15, 2024) filed in this application is herein incorporated by reference in its entirety.

### FIELD OF THE INVENTION

The present invention relates to methods of diagnosing and monitoring disease. In particular, the present invention relates to the use of ABCF1 in diagnosing and/or monitoring inflammatory and autoimmune diseases.

### BACKGROUND

Inflammation and immune responses are tightly controlled cellular mechanisms that help maintain cellular homeostasis. These mechanisms are governed by several proteins that regulate a cascade of downstream effectors.

Macrophage polarization is a process by which macrophages adopt different functional programs in response to the signals from their microenvironment. Macrophage phenotype has been divided into 2 groups: M1 and M2. The M1 phenotype is stimulated by microbial products or pro-inflammatory cytokines including IFN-γ, TNF, or Toll-like receptor (TLR) ligands. M1 macrophages produce pro-inflammatory cytokines including but not limited to TNFα, IL-1, IL-6, IL-12, Type I IFN, CXCL1-3, CXCL-5, and CXCL8-10. M2 macrophages resolve inflammation, help tissue healing, tolerate self-antigens and certain neoantigens. M2 macrophages produce anti-inflammatory cytokines such as IL-10.

ATR-binding cassette sub-family F member 1 (ABCF1) has been associated with immune signaling and various autoimmune disorders. ABCF1 is an E2 ubiquitin-conjugating enzyme that regulates various innate immune responses in macrophages, including potentiation of TLR4 endocytosis and M2 polarization, and promotes endotoxin tolerance and survival during septic toxic shock (Arora et al., Immunity 50, 1-14, 2019).

ABCF1 acts as a ubiquitin-switch that regulates inflammatory pathways. Although ABCF1 (+/) mice appear normal under specific pathogen-free conditions, it was recently discovered that ABCF1 acts as a molecular switch between inflammatory pathways downstream of TLRs ⁵³. In the Immunity paper, “The ATP-Binding Cassette Gene ABCF1 Functions as an E2 Ubiquitin-Conjugating Enzyme Controlling Macrophage Polarization to Dampen Lethal Septic Shock” (2019) ⁵³, sepsis was studied, where little was known regarding the molecular switches and pathways that regulate this disease. It was discovered that ABCF1 possesses an E2 ubiquitin enzyme activity, through which it controls the LPS-Toll-like Receptor-4 (TLR4)-mediated gram-negative insult by targeting key proteins for K63-polyubiquitination. K63-ubiquitination by ABCF1 shifts the inflammatory profile from an early phase MyD88-dependent to a late phase TRIF-dependent signalling pathway, thereby regulating TLR4 endocytosis and modulating macrophage polarization from M1 to M2 phase. Physiologically, ABCF1 regulates the shift from the inflammatory phase of sepsis to the endotoxin tolerance phase and modulates cytokine storm and interferon-β-dependent production by the immunotherapeutic mediator, SIRT1. Consequently, ABCF1 controls sepsis-induced mortality by repressing hypotension induced renal circulatory dysfunction. Further, ABCF1 is necessary to maintain macrophage polarization in M2b state and the lack of ABCF1 shifts the state to the pro-inflammatory M1 state 53.

The molecular details of the ABCF1 switch are as follows. In the MyD88 pathway (M1 macrophage-like), the early phase of TLR4 signalling leads to UBC13 targeting TRAF6 for K63-polyubiquitination, which further targets cIAP1/2 for K63-polyubiquitination. cIAP1/2 then enhances K48-proteasomal degradation of ABCF1 and TRAF3. In the absence of ABCF1, TAK1 is phosphorylated, which leads to activation of MARK and NF-κB pathways and elevated production of pro-inflammatory cytokines like TNFα, IL-1b, IL-6, thereby polarizing macrophages to M1 phenotype. Subsequently in the TRIF pathway (M2 macrophage-like), self K48-proteasomal degradation of cIAP1/2 results in K63-polyubiquitination of ABCF1 by TRAF6, which results in ABCF1 to bind and forms a complex with TRAF3 and SYK leading to the formation of K63-polyubiquitylated TRAF3 and SYK. This leads to TLR4 endocytosis into the endosomes, which then initiates TRIF-dependent TLR4 signalling and eventual production of IFN-I stimulated genes. This triggers phosphorylation of TBK1 that leads to phosphorylation and eventual dimerization of IRF3 and production of IFN-I stimulated genes. This shift from MyD88 to TRIF signalling by ABCF1 leads to increased production of IL-10, minimal production of TNFα, IL-1b, IL-6 and CD86, MHC-II surface markers and decreased CD206 levels, thus polarizing macrophages to M2b phenotype.

Major depressive disorder (MDD), often referred to as “depression”, affects psychosocial functioning and diminishes the quality of life¹. It affects over 300 million people worldwide ² and is associated with −800,000 suicide deaths annually ³. The World Health Organization states that MDD will become the third most prevalent disease in the World by 2030 ⁴. It occurs in higher prevalence in women than in men, but the aetiology of depression remains poorly understood. It appears to be caused by both genetic and environmental factors, however, its diagnosis and management are clinically challenging because of unpredictable presentation and response to treatment⁴. Furthermore, depression remains associated with premature mortality from suicide and other illnesses⁵. A traditional hypothesis is that those living with depression have a deficiency in monoamine neurotransmitters such as serotonin and norepinephrine in the brain, however, evidence now shows that some forms of depression are associated with ongoing forms of low-grade inflammation⁶.

Subsets of depression patients have an impaired peripheral immune system, increased levels of proinflammatory cytokines that can affect neurotransmitter metabolism, neuroendocrine function and regional brain activity⁷. Patients given proinflammatory cytokines, such as IL-1b, experience more symptoms of anxiety and depression than untreated patients⁷, and patients experiencing bacterial and viral infections often experience symptoms associated with depression (i.e. disrupted sleep, fatigue, depressed moods, impaired concentration) 8.

Studies link MDD to higher levels of inflammatory markers compared to those who are not clinically depressed. A study of >14,000 patients showed those with depression had 46% higher levels of C-reactive protein (CRP), an inflammation marker, in their blood ⁸.

The immune balance between Th1/Th2 and Th17/Treg correlate with MDD¹⁷. Depressed subjects have an increase in peripheral Th17 cell number and a decrease in T-reg cell number resulting in imbalance of Th17/Treg ratio compared to healthy controls¹⁸. Furthermore, studies show that pregnant patients with MDD have elevated inflammatory responses ^{19,20} and higher levels of circulating steroids compared to healthy pregnant women ²¹. Specifically women exhibiting severe depression (SD) and severe anxiety (SA) during pregnancy exhibit high levels of Th1-(IL-6, TNF-α, IL-2, IFN-γ), Th 17-(IL-17A, IL-22), and Th2-(IL-9, IL-10, and IL-13) related cytokines. The SA group alone showed higher concentrations of Th1-(IL-6, TNF-α, IL-2, IFN-γ) and Th2-(IL-4, and IL-10) cytokines versus the controls¹⁷.

Moreover, the immune balance between M1/M2 macrophages has previously been proposed as a target of therapy for MDD²². Studies on humans and animals have documented that chronic activation of M1 microglial cells ²³⁻²⁵may trigger mood disorders ²⁶ through the release of a variety of chemokines, eicosanoids, free radicals, neurotoxins, pro-inflammatory cytokines, and nitric oxide ²³, thereby potentiating neuronal dysfunction²⁷. Various bacterial and viral infections including influenza virus, Herpes viruses, and HIV induce the secretion of proinflammatory cytokines and induce microglial activation that is associated with depression symptoms ²⁸⁻³². Experimental induction in humans with immune activators that activate microglia such as endotoxin (LPS) a key driver of SIRS or gram-negative bacteria such as Salmonella typhimurium induces depressive symptoms, where the severity is correlated with elevated blood levels of inflammatory cytokines³³⁻³⁵. In animal models, LPS administration induces microglial activation together with depression symptoms in rodents that is halted with selective serotonin reuptake inhibitors (SSRIs) or tricyclic antidepressants (TCAs) ^{36,37} In fact, many observations support the involvement of microglia in LPS-induced depression: (i) LPS-induced depression symptoms can be reduced by treatment with the microglial inhibitor minocycline 38; (ii) activation of the enzyme indoleamine 2,3-dioxygenase (IDO) in microglia is essential for the development of depression symptoms and microglial activation induced by LPS ³⁸⁻⁴¹, and (iii) mice with microglial hyper-reactivity by traumatic brain injury 42, or induced by a microglia-specific mutation in the fractalkine receptor ⁴³ exhibit heightened LPS-induced depression symptoms. In contrast mice deficient in NLRP3 inflammasome signalling resulting in induction of pro-inflammatory cytokine secretion have attenuated depression in response to LPS ⁴⁴.

ABCF1 is a missing link in inflammatory disease and depression. Gene expression of ABCF1 has been shown to be elevated substantially in human synoviocytes isolated from the inflamed joints of rheumatoid arthritis patients, and this increases further when stimulated with TNF-α ⁵⁰. Also, the ABCF1 locus is linked to increased susceptibility to autoimmune pancreatitis in the Japanese population ⁵¹ and, has been associated with susceptibility to rheumatoid arthritis in European and Asian populations ⁵².

Escitalopram, an antidepressant of the SSRI (selective serotonin receptor inhibitor) class, has been reported to influence anti-inflammatory pathways in patient populations and it was concluded that ABCF1 is Escitalopram's putative therapeutic target.

KR101574766B1 teaches a biomarker composition for diagnosing Alzheimer's disease which includes ABCF1 in CSF. In addition, it is known in the art that chronic inflammation plays a role in Alzheimer's disease and ABCF1 expression is higher in APP/PS1 mice as compared to Wild type mice (Jorda et al. Int. J. Biol. Sci 2019 15 (2): 453-463.

### SUMMARY OF THE INVENTION

An object of the present invention is the use of ABCF1 in methods of diagnosing and monitoring inflammatory and/or autoimmune disease. In one aspect of the present invention, there is provided a method of monitoring an inflammatory or immune response, said method comprising a) collecting samples at pre-determined intervals from a subject; b) measuring the level of ABCF1 in each of the samples collected from the patient; and c) comparing the level of ABCF1 in each of the samples; wherein a decrease in the level of ABCF1 over time indicates that the inflammatory or immune response is increasing and wherein an increase in the level of ABCF1 over time indicates that the inflammatory or immune response is decreasing.

In another aspect of the present invention, there is provided a method of diagnosing a disease or disorder, optionally associated with inflammatory and/or autoimmune diseases in a patient comprising the steps of: a) collecting a sample from the patient; b) measuring the level of ABCF1 in the sample collected from the patient; and c) comparing the levels of ABCF1 with a predefined level of ABCF1; wherein a correlation between the ABCF1 level in the patient sample and predefined ABCF1 levels indicates that the patient has said disease or disorder.

In another aspect of the present invention, there is provided a method of monitoring progression of a disease, optionally an inflammatory and/or autoimmune disease, the method comprising a) collecting samples at pre-determined intervals from a subject having said disease; b) measuring the level of ABCF1 in each of the samples collected from the patient; c) comparing the level of ABCF1 in each of the samples; wherein a decrease in the level of ABCF1 over time indicates that the disease is worsening and wherein an increase in the level of ABCF1 over time indicates that the disease is improving.

In another aspect of the present invention, there is provided a method of monitoring treatment of a disease, optionally an inflammatory and/or autoimmune disease, the method comprising a) collecting samples at pre-determined intervals from a subject having said disease and undergoing the treatment; b) measuring the level of ABCF1 in each of the samples collected from the patient; c) comparing the level of ABCF1 in each of the samples; wherein a decrease in the level of ABCF1 during the treatment indicates that the the treatment is not effective and wherein an increase in the level of ABCF1 over time indicates the treatment is effective.

In certain embodiments, the disease is an inflammatory and/or autoimmune diseases selected from arthritis, including but not limited to Rheumatoid Arthritis and other autoimmune arthritis, thyroid autoimmune diseases including but not limited to Grave's disease, cutaneous lupus erythematosus, autoimmune pancreatitis, inflammatory bowel disease including but not limited to Crohn's disease and ulcerative colitis and neuroinflammatory diseases and disorders including but not limited to Major Depressive Disorder (MDD) and multiple sclerosis (MS).

In certain embodiments, the disease or disorder is sepsis, including but not limited to preeclampsia sepsis.

In certain embodiments, the disease is postpartum depression.

In certain embodiments, the disease is Major Depressive Disorder.

In certain embodiments, the disorder is addiction, addictive behaviour, compulsive disorder or inability to concentrate.

In certain embodiments, wherein the disease or disorder is post-COVID-19.

In certain embodiments, wherein the disorder is a neurological disorder post-COVID-19.

In certain embodiments, wherein the disorder is post-COVID-19 brain fog.

In certain embodiments, ABCF1 and optionally other markers is measured using mass spectrometry or an immunoassay.

In certain embodiments, circulating biomarker DNA or RNA is measured.

### BRIEF DESCRIPTION OF THE FIGURES

These and other features of the invention will become more apparent in the following detailed description in which reference is made to the appended drawings.

FIG. 1 illustrates that Escitalopram induces ABCF1 in a Macrophage cell line: RAW macrophages were plated at 1×10⁵ cells/well and cultured for 2 days. The cells were incubated with 0.3 mM Escitalopram for 1 hour, and then harvested for total RNA, which was extracted for real time RT-PCR specific for ABCF1 and IL-4. CT values were normalized with CT value for the housekeeping gene from the DMSO control. The difference in the expression after drug treatment is consistent with polarization towards an M2-like phenotype (data were consistent in 3 separate experiments).

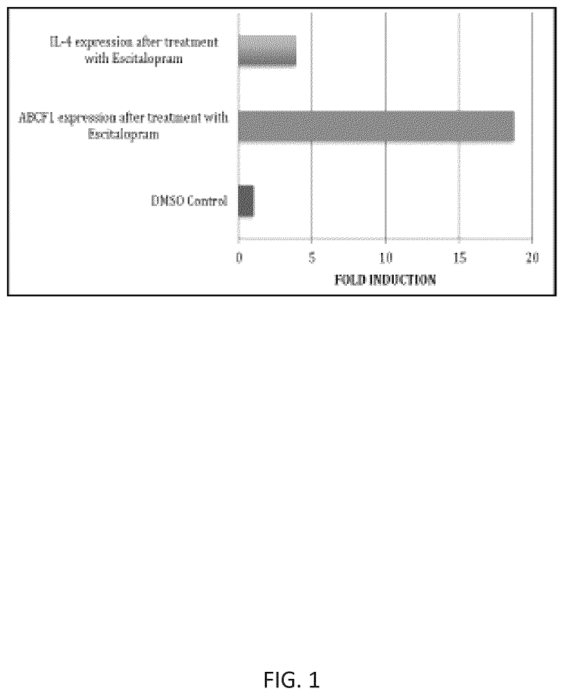

FIG. 2 illustrates image acquisition and data analysis using Cellomics Arrayscan V^{TI}. (A) The Hoechst channel shows the Hoechst stained nuclei of LMD:TAP-1 cells. (B) The GFP channel shows the cytoplasmic GFP expression in LMD:TAP-1 cells. During image analysis, an algorithm is applied to identify the nuclei based on Hoechst fluorescence intensity (C), apply a cytoplasmic mask (D) and quantitate GFP fluorescence intensity within the cytoplasmic mask area.

FIG. 3 illustrates the effect of psylocibin, psylocin and their analogs on ABCF1 transcription in a macrophage cell line. Briefly, the Macrophage cell line RAW264.7 (ATCC) were grown to 80% confluency in growth media (DMEM+ 10% FBS+ glutamine). Dilutions of psylocibin, psylocin and their analogs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole. The cells were incubated for 2 hours and then harvested for total RNA, which was extracted for real time RT-PCR specific for ABCF1. Untreated cells were used as negative control and Escitalopram at 0.3 mM was used as a positive control to activate ABCF1 expression for all the experiments. ES=escitalopram; PSYB=Psylocibin; PSIC=Psilocin; DMT=4-Acetoxy-N, N-dimthyltryptamine; APF=O-Acetyl Psilocin Fumerate, and AOI=4-acetoxyindole.

### DETAILED DESCRIPTION

The present invention, is based on the discovery that ABCF1 is a strong negative regulator of pro-inflammatory responses and changes in ABCF1 activity/expression is associated with a number of inflammatory and/or autoimmune diseases. ABCF1 mediates M2 polarization. The M1 phenotype is stimulated by microbial products or pro-inflammatory cytokines [IFN-γ, TNF, or Toll-like receptor (TLR) ligands]. M1 macrophages produce pro-inflammatory cytokines including but not limited to TNFα, IL-1, IL-6, IL-12, Type I IFN, CXCL1-3, CXCL-5, and CXCL8-10. M2 macrophages resolve inflammation, help tissue healing, tolerate self-antigens and certain neoantigens. M2 macrophages produce anti-inflammatory cytokines such as IL-10. A decrease in activity/expression of ABCF1 may result in M1 polarization and an increased inflammatory response while an increase in activity/expression of ABCF1 may result in M2 polarization and a decreased inflammatory response.

Accordingly, ABCF1 may be used as a biomarker alone or in combination with other for diagnosing and monitoring inflammatory responses and/or disease progression/treatment inflammatory and/or autoimmune diseases.

In certain embodiments, ABCF1 is part of a panel of biomarkers for diagnosing and monitoring inflammatory responses and/or disease progression/treatment inflammatory and/or autoimmune diseases. The other biomarkers may include but are not limited to for example cytokines such as interleukin (IL)-1B, IL-6, IL-10, monocyte chemoattractant protein-1, tumor necrosis factor-alpha and inflammatory markers such as C-reactive protein, and phospholipase A2. Further biomarkers may include but are not limited to alpha-1-add glycoprotein, C-reactive protein, Complement C3, Fibrinogen gamma chain, Haptoglobin, Immunoglobulin G total, Immunoglobulin M, L-plastin, LPS binding protein, Mannose Binding Lectin, Myleoperoxidase and Serum amyloid A1.

The ABCF1 protein and nucleic acid sequences (genomic and cDNA) are known in the art. See for example GenBank Accession numbers AQY76226.1, AQY76225.1, KY500135.1 and KY500134.1. In certain embodiments, the ABCF1 comprises the sequence set forth below:

(SEQ ID NO: 1)
MPKAPKQQPPEPEWIGDGESTSPSDKVVKKGKKDKKIKKTFFEELAVEDKAGEEEKVLK
EKEQQQQQQQQQQKKKRDTRKGRRKKDVDDDGEEKELMERLKKLSVPTSDEEDEVPAP
KPRGGKKTKGGNVFAALIQDQSEEEEEEEKHPPKPAKPEKNRINKAVSEEQQPALKGKKG
KEEKSKGKAKPQNKFAALDNEEEDKEEEIIKEKEPPKQGKEKAKKAEQGSEEEGEGEEEEE
EGGESKADDPYAHLSKKEKKKLKKQMEYERQVASLKAANAAENDFSVSQAEMSSRQAM
ENASDIKLEKFSISAHGKELFVNADLYIVAGRRYGLVGPNGKGKTTLLKHIANRALSIPPNID
VLLCEQEVVADETPAVQAVLRADTKRLKLLEEERRLQGQLEQGDDTAAERLEKVYEELRA
TGAAAAEAKARRILAGLGFDPEMQNRPTQKFSGGWRMRVSLARALFMEPTLLMLDEPTN
HLDLNAVIWLNNYLQGWRKTLLIVSHDQGFLDDVCTDIIHLDAQRLHYYRGNYMTFKKM
YQQKQKELLKQYEKQEKKLKELKAGGKSTKQAEKQTKEALTRKQQKCRRKNQDEESQEA
PELLKRPKEYTVRFTFPDPPPLSPPVLGLHGVTFGYQGQKPLFKNLDFGIDMDSRICIVGPNG
VGKSTLLLLLTGKLTPTHGEMRKNHRLKIGFFNQQYAEQLRMEETPTEYLQRGFNLPYQD
ARKCLGRFGLESHAHTIQICKLSGGQKARVVFAELACREPDVLILDEPTNNLDIESIDALGE
AINEYKGAVIVVSHDARLITETNCQLWVVEEQSVSQIDGDFEDYKRE
VLEALGEVMVSRPRE

Nucleic add probes targeting ABCF1 mRNA are known in the art. Anti ABCF1 antibodies are known in the art and are available commercially.

ABCF1 peptides are known in the art and include but are not limited to

(SEQ ID NO: 2)
QQPPEPEWIG DGESTSPSDK VVK;
(SEQ ID NO: 3)
QQPPEPEWIG DGESTSPSDK VVKK;
(SEQ ID NO: 4)
LKKLSVPTSD EEDEVPAPKP R;
(SEQ ID NO: 5)
KLSVPTSDEE DEVPAPKPR;
(SEQ ID NO: 6)
FAALDNEEED KEEEIIKEKE PPKQGKEK;
(SEQ ID NO: 7)
KAEQGSEEEG EGEEEEEEGG ESKADDPYAHLSK;
(SEQ ID NO: 8)
KAEQGSEEEG EGEEEEEEGG ESKADDPYAH LSKK;
(SEQ ID NO: 9)
AANAAENDFS VSQAEMSSR;
(SEQ ID NO: 10)
QAMLENASDI KLEK;
(SEQ ID NO: 11)
ELFVNADLYI VAGR;
(SEQ ID NO: 12)
ELFVNADLYI VAGRR;
(SEQ ID NO: 13)
ALSIPPNIDV LLCEQEVVAD ETPAVQAVLR;
(SEQ ID NO: 14)
RLQGQLEQGD DTAAERLEK;
(SEQ ID NO: 15)
RLQGQLEQGD DTAAERLEKV YEELR;
(SEQ ID NO: 16)
VYEELRATGA AAAEAK;
(SEQ ID NO: 17)
RILAGLGFDP EMQNRPTQK;
(SEQ ID NO: 18)
TLLIVSHDQG FLDDVCTDII HLDAQR;
(SEQ ID NO: 19)
KNQDEESQEA PELLKR;
(SEQ ID NO: 20)
KNQDEESQEA PELLKRPK;
(SEQ ID NO: 21)
NQDEESQEAP ELLK;
(SEQ ID NO: 22)
NQDEESQEAPELLKRPK;
(SEQ ID NO: 23)
FTFPDPPPLS PPVLGLHGVT FGYQGQK;
(SEQ ID NO: 24)
FTFPDPPPLS PPVLGLHGVT FGYQGQKPLF K;
(SEQ ID NO: 25)
NLDFGIDMDS R;
(SEQ ID NO: 26)
ICIVGPNGVG K;
(SEQ ID NO: 27)
STLLLLLTGK;
(SEQ ID NO: 28)
STLLLLLTGKLTPTHGEMR;
(SEQ ID NO: 29)
LKIGFFNQQY AEQLRMEETP TEYLQR;
(SEQ ID NO: 30)
IGFFNQQYAE QLR;
(SEQ ID NO: 31)
IGFFNQQYAE QLRMEETPTE YLQR;
(SEQ ID NO: 32)
MEETPTEYLQ R;
(SEQ ID NO: 33)
GFNLPYQDAR;
(SEQ ID NO: 34)
GFNLPYQDAR K;
(SEQ ID NO: 35)
FGLESHAHTI QICK;
(SEQ ID NO: 36)
VVFAELACRE PDVLILDEPT NNLDIESIDA LGEAINEYK;
(SEQ ID NO: 37)
EPDVLILDEP TNNLDIESID ALGEAINEYK GAVIVVSHDA R;
(SEQ ID NO: 38)
LITETNCQLW VVEEQSVSQI DGDFEDYKR;
(SEQ ID NO: 39)
EVLEALGEVM VSRPR;
(SEQ ID NO: 40)
EVLEALGEVM VSRPRE.

There are secreted and cell retained forms of ABCF1. Accordingly, in certain embodiments, both forms are measured, in certain embodiments, secreted ABCF1 is measured. In certain embodiments, the cell retained form is measured.

In certain embodiments, ABCF1 expression levels is measured in a biological sample collected from a subject. As used herein, a “biological sample” (also referred to as a “sample”) includes but is not limited to blood, plasma, serum, urine, sweat, cerebrospinal fluid, pleural fluid, bronchial lavages, sputum, peritoneal fluid, bladder washings, secretions (e.g., breast secretions), oral washings, swabs (e.g., oral swabs), isolated cells, tissue samples, touch preps, and fine-needle aspirates. In certain embodiments, the biological sample is a serum sample. In certain embodiments, the biological sample is a urine sample. In certain embodiments, the biological sample is a CSF sample. In certain embodiments, the biological sample is a cell sample. In specific embodiments, the biological sample is a peripheral blood mononuclear cell (PBMC) sample.

In some embodiments, if a biological sample is to be tested immediately, the sample may be maintained at room temperature. In other embodiments, the sample may be refrigerated or frozen (e.g., at −80° C.) prior to assay.

In certain embodiments, multiple samples are collected from the subject at different times. For example, samples may be collected before, during or following treatment with a pharmaceutical or biologically active substance or at regularly scheduled intervals. In certain embodiments, comparing the expression/level of ABCF1 alone or in combination with other biomarkers in samples obtained at different times may be used to monitor disease progression and/or the effectiveness of a treatment regime.

Methods of measuring gene expression including mRNA and protein expression are known in the art. For example, mRNA may be measured using Northern blots, quantitative Reverse Transcription PCR (qRT-PCR) and microarrays. Protein expression may be measured using mass spectrometry including but not limited to SISCAPA (Stable Isotope Standards and Capture by Anti-Peptide Antibodies mass spectrometry (MS) and liquid-chromatography mass spectrometry (LC-MS) and immunoassays including but not limited to Enzyme-Linked Immunosorbent Assay (ELISA), Western-blotting, and immunoarrays.

In certain embodiments, circulating biomarker nucleic adds measured. Circulating nucleic acids are any type of DNA or RNA that is present in body biofluids, including but not limited to plasma, serum and urine. They can be found within extracellular vesicles or as cell-free DNA and RNA. Several technical approaches for the analysis circulating nucleic acids are known in the art and include quantitative polymerase chain reaction (qPCR); quantitative methylation-specific PCR; droplet digital PCR; bisulfite droplet digital PCR; targeted DNA sequencing; whole-exome sequencing; whole-genome sequencing; whole-genome methylation sequencing; and beads, emulsion, amplification, and magnetics (BEAMing).

In certain embodiments, decreased expression of ABCF1 is indicative of an inflammatory and/or immune response or indicates that an inflammatory and/or immune response is/will be increasing. In certain embodiments, increased expression of ABCF1 is indicative of a decreased inflammatory and/or immune response or indicates that an inflammatory and/or immune response is/will be decreasing/resolving. The level of ABCF1 in a sample may be compared to standard level of ABCF1 and a level of ABCF1 below the standard level is indicative of an increasing inflammatory and/or immune response. In certain embodiments, expression of ABCF1 and other biomarkers of inflammation are determined.

Accordingly, monitoring expression of ABCF1 alone or in combination with other biomarkers of inflammation may be used to monitor an inflammatory and/or immune response. In certain embodiments, there is provided a method of monitoring an inflammatory or immune response, said method comprising a) collecting samples at pre-determined intervals from a subject; b) measuring the level of ABCF1 (and optionally other biomarkers of inflammation) in each of the samples collected from the patient; c) comparing the level of ABCF1 (and optionally other biomarkers of inflammation) in each of the samples; wherein a decrease in the level of ABCF1 and optionally an increase in pro-inflammatory biomarkers and/or a decrease in anti-inflammatory biomarkers over time indicates that the inflammatory or immune response is increasing and wherein an increase in the level of ABCF1 and optionally an decrease in pro-inflammatory biomarkers and/or an increase in anti-inflammatory biomarkers over time indicates that the inflammatory or immune response is decreasing.

In certain embodiments, there is provided a method of diagnosing an inflammatory and/or autoimmune disease in a patient comprising the steps of: a) collecting a sample from the patient; b) measuring the level of ABCF1 and optionally other biomarkers in the sample collected from the patient; and c) comparing the levels of ABCF1 and optionally other biomarkers with a predefined level of ABCF1, wherein a correlation between the ABCF1 level and optionally other biomarker levels in the patient sample and predefined ABCF1 (and optionally other biomarker) levels indicates that the patient has an inflammatory and/or autoimmune disease. In certain embodiments, additional biomarkers are measured. In certain embodiments, the method of diagnosis further comprises an analysis of patient symptoms.

ABCF1 expression may also be used in methods of monitoring progression of an inflammatory and/or autoimmune diseases.

In certain embodiments, there is provided a method of monitoring progression of an inflammatory and/or autoimmune disease, the method comprising a) collecting samples at pre-determined intervals from a subject having said inflammatory and/or autoimmune disease; b) measuring the level of ABCF1 and optionally other biomarkers in each of the samples collected from the patient; c) comparing the level of ABCF1 and optionally other biomarkers in each of the samples; wherein a change in the level of ABCF1 and optionally a change in pro-inflammatory and/or anti-inflammatory biomarkers indicates that the inflammatory and/or autoimmune disease is changing over time.

In specific embodiments, a decrease in the level of ABCF1 and optionally an increase in pro-inflammatory biomarkers and/or a decrease in anti-inflammatory biomarkers over time indicates that the inflammatory disease is worsening and wherein an increase in the level of ABCF1 and optionally an decrease in pro-inflammatory biomarkers and/or an increase in anti-inflammatory biomarkers over time indicates that the inflammatory is improving.

In specific embodiments for autoimmune diseases where M1 macrophages play a role in pathogenesis, a decrease in the level of ABCF1 and optionally an increase in pro-inflammatory biomarkers and/or a decrease in anti-inflammatory biomarkers over time indicates that the autoimmune disease is worsening and wherein an increase in the level of ABCF1 and optionally an decrease in pro-inflammatory biomarkers and/or an increase in anti-inflammatory biomarkers over time indicates that the autoimmune disease is improving.

In specific embodiments for autoimmune diseases where M2 macrophages play a role in pathogenesis, an increase in the level of ABCF1 over time indicates that the autoimmune disease is worsening and wherein an increase in the level of ABCF1 and optionally an decrease in pro-inflammatory biomarkers and/or an increase in anti-inflammatory biomarkers over time indicates that the autoimmune disease is improving.

In certain embodiments, additional biomarkers are measured. In certain embodiments, the method of diagnosis further comprises an analysis of patient symptoms.

ABCF1 expression may also be used in methods of monitoring treatment of an inflammatory and/or autoimmune diseases. In particular, the present invention provides methods for determining the efficacy of a particular therapy, including a particular drug or combination of drugs for treatment of an inflammatory and/or autoimmune disease. These methods are useful in performing clinical trials of a particular therapy or drug, as well as monitoring the progress of a patient undergoing the treatment. In certain embodiments, there is provided a method of monitoring treatment, the method comprising a) collecting samples at pre-determined intervals from a subject having said inflammatory and/or autoimmune disease and undergoing the treatment; b) measuring the level of ABCF1 and optionally other biomarkers in each of the samples collected from the patient; c) comparing the level of ABCF1 and optionally other biomarkers in each of the samples; wherein a change in ABCF1 levels is indicates that the treatment is effective. In certain embodiments for treatment of inflammatory diseases, a decrease in the level of ABCF1 and optionally an increase in pro-inflammatory biomarkers and/or a decrease in anti-inflammatory biomarkers during the treatment indicates that the treatment is not effective and wherein an increase in the level of ABCF1 and optionally a decrease in pro-inflammatory biomarkers and/or an increase in anti-inflammatory biomarkers over time indicates that the treatment is effective.

In specific embodiments for autoimmune diseases where M1 macrophages play a role in pathogenesis, a decrease in the level of ABCF1 and optionally an increase in pro-inflammatory biomarkers and/or a decrease in anti-inflammatory biomarkers over time indicates that the autoimmune disease is worsening and wherein an increase in the level of ABCF1 and optionally an decrease in pro-inflammatory biomarkers and/or an increase in anti-inflammatory biomarkers over time indicates that the treatment is effective.

In specific embodiments for autoimmune diseases where M2 macrophages play a role in pathogenesis, an increase in the level of ABCF1 over time indicates that the autoimmune disease is worsening and wherein an increase in the level of ABCF1 and optionally an decrease in pro-inflammatory biomarkers and/or an increase in anti-inflammatory biomarkers over time indicates that the treatment is effective.

In certain embodiments, additional biomarkers are measured. In certain embodiments, the method of diagnosis further comprises an analysis of patient symptoms.

Exemplary inflammatory and/or autoimmune diseases that ABCF1 may be used as a biomarker for include but are not limited to arthritis, including but not limited to Rheumatoid Arthritis and other autoimmune arthritis, thyroid autoimmune diseases including but not limited to Grave's disease, cutaneous lupus erythematosus, autoimmune pancreatitis, inflammatory bowel disease including but not limited to Crohn's disease and ulcerative colitis and neuroinflammatory diseases and disorders including but not limited to Major Depressive Disorder (MDD) and multiple sclerosis (MS). Inflammatory diseases may also include infectious diseases including but not limited to viral and bacterial diseases.

ABCF1 regulates dsDNA-induced immune responses in human epithelial cells (Cao et al. Front. Cell. Infect. Microbiol., 16 Sep. 2020 https://doi.org/10.3389/fcimb.2020.00487.

Infection with SARS-COV-2 (COVID19) has been shown to upregulate cytokines, chemokines and interferon which results in a hyperinflammatory state. In addition, the severity of SARS-COV-2 infection has been shown to be associated with dysregulation of inflammatory immune responses.

Severe COVID-19 is caused by a severe innate immune system inflammatory reactions resulting in a cytokine storm. A cytokine storm is an activation cascade of auto-amplifying cytokine production due to unregulated host immune response to different triggers. Expression of ABCF1 in postmortem lung samples show decreased expression in COVID patients (https://covidgenes.weill.cornell.edu/).

Accordingly, in certain embodiments, ABCF1 is used alone or as part of a panel of biomarkers for predicting the prognosis of COVID-19.

In certain embodiments ABCF1 is used alone or as part of a panel of biomarkers for diagnosing and monitoring inflammatory and/or autoimmune diseases. In specific embodiments, the inflammatory and/or autoimmune diseases are selected from arthritis, including but not limited to Rheumatoid Arthritis and other autoimmune arthritis, thyroid autoimmune diseases including but not limited to Grave's disease, cutaneous lupus erythematosus, autoimmune pancreatitis, inflammatory bowel disease including but not limited to Crohn's disease and ulcerative colitis and neuroinflammatory diseases and disorders including but not limited to Major Depressive Disorder (MDD), multiple sclerosis (MS) and COVID-19.

In specific embodiments ABCF1 is used alone or as part of a panel of biomarkers for diagnosing and monitoring Major Depressive Disorder (MDD) and/or Major Depressive Disorder (MDD) progression/treatment including but not limited to treatment with Selective serotonin reuptake inhibitors (SSRIs).

In specific embodiments ABCF1 is used alone or as part of a panel of biomarkers for diagnosing and monitoring COVID-19 and/or COVID-19 progression/treatment.

Approximately one-third of patients with COVID-19 develop neurological symptoms (e.g. COVID brain fog). B-Amyloid deposits have been found in young COVID-19 patients. ABCF1 appears to directly interact with Beta-amyloid (https://thebiogrid.org/106541/summary/homo-sapiens/abcf1.html). Accordingly, in certain embodiments, ABCF1 may be used to determine risk of developing COVID neurological symptoms.

In specific embodiments, ABCF1 is part of a panel of biomarkers. In specific embodiments, the panel further comprises one or more of the following biomarkers interleukin (IL)-1B, IL-6, IL-10, monocyte chemoattractant protein-1, tumor necrosis factor-alpha, C-reactive protein, and phospholipase A2. In specific embodiments, the panel comprises ABCF1, interleukin (IL)-1ß, IL-6, IL-10, monocyte chemoattractant protein-1, tumor necrosis factor-alpha, C-reactive protein, and phospholipase A2.

In certain embodiments, the depression biomarker panel comprises one or more of COX-2, MPO, iNOS, and secretory phospholipase A2 type IIA in addition to ABCF1. These biomarkers have been shown to be increased in patients with recurrent depressive disorder. In specific embodiments, the depression biomarker panel comprises ABCF1, COX-2, MPO, iNOS, and secretory phospholipase A2 type IIA. In certain embodiments, the biomarkers panel further comprises one or more of tryptophan hydroxylase, Tph1 and Tph2. In certain embodiments, the biomarkers panel further comprises one or more of enzymes that regulate Vitamin D synthesis and/or regulate serotonin synthesis including 7-dehydrocholesterol (7-DHC) reductase, 25-hydroxylases (25-OHase, 25-hydroxyvitamin D3-1-hydroxylase (1-OHase) and 24-hydroxylase (24-OHase).

It has been previously shown that depressed patients overall had higher levels of IL-1B, IL-6, macrophage-inhibiting factor (MIF), and TNF-αr, and lower levels of the anti-inflammatory cytokine, IL-4, compared with controls. Patients who were less responsive to antidepressants also have the highest levels of IL-1β, MIF, and TNF-α, and successful antidepressant response was associated with normalization of IL-6 levels. Accordingly, in certain embodiments, the panel of biomarkers comprises IL-1β, IL-6, macrophage-inhibiting factor (MIF), and TNF-α, and IL-4 in addition to ABCF1.

In specific embodiments, there is provided a method of monitoring treatment of depression, the method comprising a) collecting samples at pre-determined intervals from a subject having depression and undergoing the treatment; b) measuring the level of ABCF1 and optionally other biomarkers in each of the samples collected from the patient; c) comparing the level of ABCF1 and optionally other biomarkers in each of the samples; wherein a decrease in the level of ABCF1 and optionally an increase in pro-inflammatory biomarkers and/or a decrease in anti-inflammatory biomarkers during the treatment indicates that the treatment is not effective and wherein an increase in the level of ABCF1 and optionally an decrease in pro-inflammatory biomarkers and/or an increase in anti-inflammatory biomarkers over time indicates the treatment is effective. In certain embodiments, additional biomarkers are measured. In certain embodiments, the method of diagnosis further comprises an analysis of patient symptoms. Symptoms of MDD include but are not limited to trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; and suicidal thoughts or attempts.

In certain embodiments, ABCF1 is used alone or as part of a panel of biomarkers for addiction, suicide, compulsive behaviour, inability to concentrate and chronic fatigue.

In another aspect, the present invention provides kits for use in the methods of the present. In a specific embodiment, the kit is provided as an ELISA kit comprising antibodies to the ABCF1. The ELISA kit may comprise a solid support, such as a chip, microtiter plate (e.g., a 96-well plate), bead, or resin having cytokine capture reagents attached thereon. The kit may further comprise a means for detecting ABCF1, such as antibodies, and a secondary antibody-signal complex such as horseradish peroxidase (HRP)-conjugated goat anti-rabbit igG antibody and tetramethyl benzidine (TMB) as a substrate for HRP.

The kit may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

The kit can also comprise a washing solution or instructions for making a washing solution, in which the combination of the capture reagents and the washing solution allows capture of the ABCF1 on the solid support for subsequent detection by, e.g., antibodies or mass spectrometry. In a further embodiment, a kit can comprise instructions for suitable operational parameters in the form of a label or separate insert. For example, the instructions for use. In yet another embodiment, the kit can comprise one or more containers with ABCF1 samples, to be used as standard(s) for calibration.

In certain embodiments, there is provided methods to identity agents including drugs and natural extracts that modulate ABCF1 expression and therefore may be useful in the identification of drugs. In specific embodiments, a reporter gene is placed under the control of the ABCF1 promoter and the reporter gene product is measured (either qualitatively or quantitatively). Cells, including but not limited macrophages such as RAW 264.7 cell line, comprising the ABCF1 promoter reporter gene product may be used in assays to identity agents that modulate ABCF1 expression.

Such methods may be useful in the identification of drugs to treat depression and anti-inflammatory drugs. Such methods may also be used to exclude drugs or extracts that may negatively effect depression or mood disorders

In certain embodiments, the U937 cell line is used to screen for agents that impact M1 M2 polarization.

### EXAMPLES

### Example 1: Innate and Adaptive Immunity in the Pathophysiology and Treatment of Depression

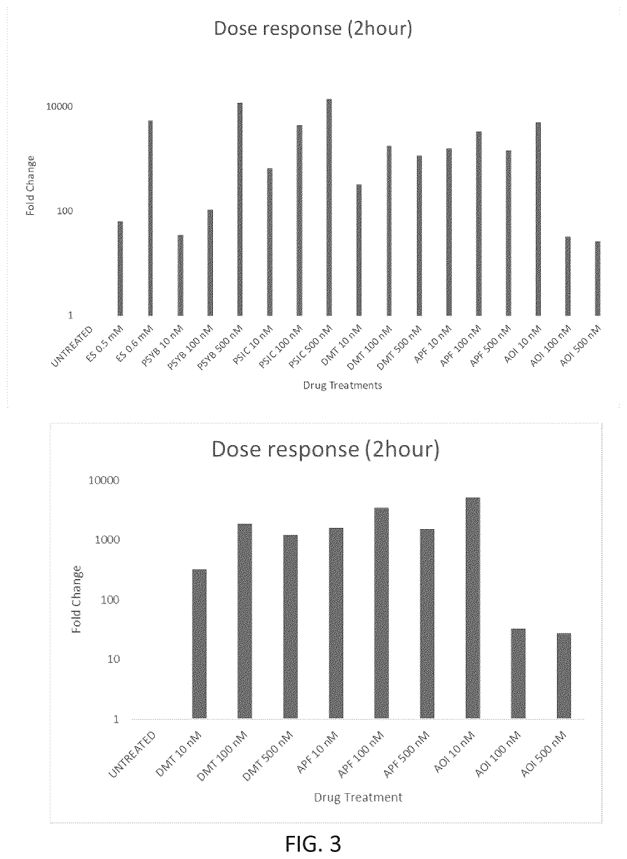

Escitalopram is a selective serotonin reuptake inhibitor (SSRI) and has the highest selectivity for the serotonin transporter compared to the norepinephrine transporter, making the side-effect profile relatively mild in comparison to less selective SSRIs⁵⁴. Additionally, noradrenergic or serotonin-norepinephrine reuptake inhibitors used to treat major depressive disorder have anti-inflammatory properties in vitro⁵⁵. Antidepressants, such as escitalopram, appear to possess anti-inflammatory properties^{56,57,58-60}. Mechanistically, antidepressants likely mediate this through a reduction in pro-inflammatory cytokines IL-1b, TNFα, and IL-6 with a reciprocal increase in anti-inflammatory cytokines including IL-10a. Studies have also shown that single nucleotide polymorphisms in IL-6 and IL-11, and mRNA levels of TNFα, are predictive of clinical response to the SSRI, escitalopram^{63,64}. Also, escitalopram modulates mRNA levels of cytokines in mouse brain⁶⁵ and decreases cytokine mRNA levels in the circulating immune cells of depression patients⁶⁰. Furthermore, IL-6 mRNA levels correlate to clinical response in depressed patients treated with antidepressants⁶⁰, and several cytokines, including IL-1 b and TNFα, acutely stimulate serotonin transporter activity in neurons. The alteration of transport activity in serotonergic neurons in the brain provides a mechanism by which cytokines can modulate serotonergic signaling, and subsequently influence emotional cognitive processing. Experimental induction in humans with immune activators, such as LPS that activate macrophages and microglia, act as key drivers of depression and reveal that the severity of depressive symptoms is correlated with elevated blood levels of pro-inflammatory cytokines ³³⁻³⁵. Recently, ABCF1 was identified as a putative therapeutic target of escitalopram⁶⁶. In conjunction with the Genome-Based Therapeutic Drugs for Depression Project, published with the title “ABCF1 is identified as a putative therapeutic target of escitalopram in the inflammatory cytokine pathway,” the authors found that the peripheral blood mononuclear cells (PBMCs) of patients responding to Escitalopram treatment subsequently increased the transcription of a single gene: ABCF1⁶⁶. Therefore, the effectiveness of a commonly used selective serotonin reuptake inhibitor correlates with ABCF1 expression. Furthermore, to confirm the observation that ABCF1 is elevated in the PBMCs of MDD patients is observable in vitro as well it was found that Escitalopram induces IL-4 by approximately 5 fold and ABCF1 by approximately 20-fold in the macrophage cell line consistent with polarization towards an M2 phenotype (FIG. 3). Thus, ABCF1 may be a therapeutic target as it appears to reside at the intersection between inflammatory diseases and psychiatric illness.

### Example 2: The Effect of Psylocibin, Psylocin and their Analogs on ABCF1 Transcription

### Preparation of Cells:

1. Macrophage cell line RAW264.7 (ATCC) were grown to 80% confluency in growth media (DMEM+10% FBS+glutamine).
2. Dilutions of the drugs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole.
3. A time course response experiment was done for the above stated drugs at concentrations mentioned above (10 nM, 100 nM, 500 nM). The time points chosen were 0, 30 mins, 2 hours and 24 hour.
4. Untreated cells were used as negative control and Escitalopram at 0.3 mM was used as a positive control to activate ABCF1 expression for all the experiments.
   Analysis by qPCR:

Primers used:
GAPDH FP:
(SEQ ID NO: 41)
TGGATTTGGACGCATTGGTC
GAPDH RP:
(SEQ ID NO: 42)
TTTGCACTGGTACGTGTTGAT
ABCF1 FP:
(SEQ ID NO: 43)
AGAAAGCCCGAGTTGTGTTTG
ABCF1 RP:
(SEQ ID NO: 44)
GCCCCCTTGTAGTCGTTGATG

1. Post treatment with drugs at different time points, the reaction was stopped by removing the media with the drug. Cells were then collected and RNA was isolated from these.
2. After checking the quality of the RNA, cDNA was generated and qPCR was run with ABCF1 primers as the target gene and GAPDH as the house keeping gene.
3. Normalized against the expression level of GAPDH, the fold change expression level of ABCF1 was calculated and tabulated for all treatment conditions.

The results as set forth in FIG. 4 show psylocibin, psylocin and their analogs upregulate ABCF1 transcription.

### Example 3: Screening Assays for Agents that Impact Macrophage Polarization

The human monocytic cell line, U937 (ATCC, #85011440), will be used as the cell line for screening as it has previously been shown to be capable of transitioning to either M1 or M2, depending on the external stimuli. Two vectors will be transfected into the U937 cells (either IL-1+IL-10; or IL-6+IL-10; to be known as U937: 1/10 or U937: 6/10) thereby allowing for the possibility to screen for macrophage polarization by reporter color. As both red and green vectors contain the same resistance marker, the U937 cells will be separated into clones which will be differentiated and polarized as previously described in order to confirm transfection with both vector systems. An ABCF1 promoter construct will be transfected separately into U937 cells (U937: ABCF1) and screened separately from the cytokine constructs.

Differentiation of stable U937: 1/10 or U937: ABCF1 transfected cells into macrophages will be done by culturing at 2×10⁵ cells/ml for three days in complete RPMI containing kanamycin, with or without 50 nM phorbol-12-myristate-13-acetate (PMA), as previously described ⁷⁴. Upon treatment with PMA, cells undergo growth arrest and a change of morphology owing to their attachment to the substrate. The differentiation to macrophages will be confirmed using immunofluorescence flow cytometry by measuring the increased surface expression of CD11b (Bear-1 antibody; ab36939, Abcam). For polarization studies, U937 stable transfectants will be differentiated to macrophages with PMA as above, but adding 25 mg/ml of LPS (for MI polarization) ⁷⁴ or 2 ng/ml IL-10 (for M2 polarization) 76 for the last 12 h of PMA treatment. Supernatants will be removed and kept for further analysis.

The treated cells would then subjected to PBS-EDTA (0.05 M EDTA) for 5 min, followed by gentle scraping to release and separate the cells for flow cytometry. Only clones that express green upon LPS stimulation, and red upon IL-10 stimulation will be further tested for the expression of M1 and M2 protein markers using flow cytometry: e.g. CD86, CD32, CCR7 for M1 phenotype; CD14, CD163, CCR2 for M2 phenotype 75. For the clones that pass through and show the ability to express both green and red upon proper stimulation and the characteristic protein markers will be further verified by their cytokine profile after the LPS/IL-10/PMA treatment using the Human Cytokine Array panel A (R&D Systems), according to the manufacturer's instructions.

The U937: 1/10 and U937: ABCF1 clones showing the most distinction between the M1 vs M2 phenotype will be used for downstream screening of the agents.

Seeding and Treatment of Cells: The selected U937 clones will be seeded in 96-well plates at 3500 cells per well. Twenty-four hours after seeding, cells will be cultured in the presence of extracts, LPS, IL-10, or solvent control (e.g. PBS or DMSO). Plates will be incubated for 48 hours at 37° C. in a 5% CO₂ incubator. The medium will be removed and cells fixed with 4% (v/v) paraformaldehyde containing Hoechst 33342 (to stain nuclei). Fixed cells will be stored in PBS at 4° C. until further analysis.

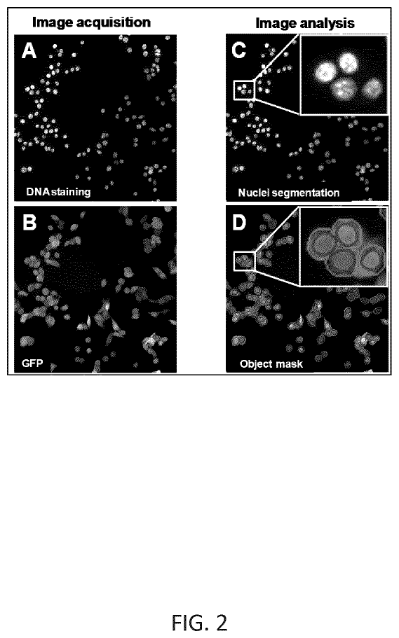

Image Analysis and Assessing the Quality of the Screening Assay: Image acquisition, segmentation and analysis of microplates will be carried out using the Cellomics™ Arrayscan V^{TI} automated fluorescence imager (FIG. 2). Image from 12 fields will be acquired using a 20× objective (auto-focus, fixed exposure time). The target activation algorithm is used to identify the nuclei based on Hoechst fluorescence intensity, apply a cytoplasmic mask and quantitate red and green fluorescence intensity within the cytoplasmic mask area. Average red and green fluorescence intensity (intensity per cell per pixel) and total number of cells per well will be determined.

To assess the quality of the screening assay, the Z′-factor will be calculated as 1−(3×δp+3×δn)/(|μp−μn|), where μp, δp, μn and δn are the means (μ) and standard deviations (s) of both the positive (p) and negative (n) controls (LPS or IL-10 and PBS alone, respectively). An assay with a Z′-factor of 0 to 0.5 is considered acceptable if the separation between the negative and positive controls is large.

Alternative Screening System Using ELISA: As an alternative to using the promoter screening tools above, U937 cells that do not contain the promoter constructs will be screen. Untransfected U937 cells will be seeded and treated as described above and then test the supernatants for the presence of secreted cytokines: for M1 polarization, we will test for IFN-γ, IL-1, IL-6, TNFα; for M2 polarization, we will test for IL-10, TGFβ, IL-4. All ELISA kits are commercially available through R&D Systems (USA).

Screening for Chemical Entities That Increase Macrophage Phagocytosis Of Cells And Bacteria: The phagocytic activity of the treated U937 macrophages will be assessed by co-culturing them with either fluorescent prostate tumour cells, bacteria (E. coli), and analyzing them for uptake of the cells/bacteria/beads using microscopy as previously described and with flow cytometry. Fluorescent beads will be used if we find the fluorescence is lost upon uptake and lysis of the cells/bacteria. Internalization of membrane antibody Fc receptors after treatment with extracts and isolated chemical entities, as compared to solvent controls can also be examined.

Screening for Chemical Entities That Increase Macrophage Killing Of Bacteria: Assessment of the extracts and isolated chemical entities that polarized the cells towards the M1 phenotype for their ability to kill bacteria (i.e. E. coli or S. typhimurium), will be assessed as has been previously described ⁹⁶. Briefly, after incubation of the treated macrophages with the bacteria for increasing times (i.e. 1 hr, 2 hr, 4 hr), the macrophages will be lysed and the remaining bacteria plated on agar plates, with serial dilution. Bacterial colonies are then counted the next day to determine the kinetics of killing by the macrophages.

Screening For Growth Inhibition: We will undertake the assessment of the extracts on the growth and viability of different cell types, including bacteria (i.e. E. coli or S. typhimurium) and primary and metastatic cancer cell lines (i.e. TC1 or A9). Starting with a known concentration of live cells, the bacteria and cancer cells will be exposed to a range of concentrations of the chemical extracts that have passed through the previous pipeline, for increasing periods of time (i.e. minutes to hours to days). They will then be assessed for their ability to survive and to grow during the exposure. Bacteria will be assessed through their colony-forming abilities, while cancer cells will be assessed using flow cytometry assays (e.g. 7AAD dye to detect viability and CFSE dye to detect cell division).

Screening For Chemical Entitles That Increase Immune Functionality: To reveal if extract treatment is effective in eliciting other immune responses, i.e. adaptive killing by T cells, and reducing the growth of bacteria or cancer cells, the following experiments will be performed.

a) Functional In Vivo Cytotoxic T lymphocyte (CTL) Assay: A standard ⁵¹Cr-release cytotoxicity assay will be used to measure antigen-specific CTL lysis of compound treated versus untreated LMD cells. Target cells (LMD) will be cultured from the tumours of compound-treated and untreated mice. Effector cells will be incubated with target cells at various effector/target ratios. The specific ⁵¹Cr release will be measured by a gamma-counter (LKB Instruments, Gaitherburg, MD) and calculated with the following equation: %⁵¹Cr release=[(experimental release−spontaneous release)/(maximum release-spontaneous release)]×100%. As a control, spontaneous ⁵¹Cr release by labeled cells will be measured in the absence of CTL, and maximum release will be quantified by lysis of target cells in 2.5% Triton X-100 detergent We would expect that the compounds we have identified with M1-skewing activity might also be able to elicit killing by CTLs.

### REFERENCES

1. Fröhlich, F. Network neuroscience. (Elsevier/Academic Press, 2016).
2. Gotlib I H, H. C. Handbook of Depression. (The Guildford Press, 2009).
3. World_Health_Organization. Depression Fact Sheet, <https://www.who.int/news-room/factsheets/detail/depression> (2020).
4. Malhi, G. S. & Mann, J. J. Lancet 392, 2299-2312, doi: 10.1016/S0140-6736 (18) 31948-2 (2018).
5. Herrman, H. et al., Lancet, doi: 10.1016/S0140-6736 (18) 32408-5 (2018).
6. Cepeda, M. S., et al., Journal of Clinical Psychiatry 77, 1666-1671,
7. Zunszain, et al. in Behavioral Neurobiology of Depression and Its Treatment. Current Topics in Behavioral Neurosciences Vol. 14 Current Topics in Behavioral Neurosciences (eds P. Cowen, T. Sharp, & J. Lau) 135-151 (Springer, 2012).
8. Hasler, G. World Psychiatry: Official Journal of the World Psychiatry Association 9, 155-161 (2013).
9. Dean, J. A Keshavan, M., Asian J Psychiatr 27, 101-111 (2017).
10. Lederberg, J. A McCray, A. T. Scientist 15, 8-8 (2001).
11. Maes, M., et al. Neuroendocrine Lett 29, 117-124 (2008).
12. Maes, M., et al. J Affect Disorders 141, 55-62, (2012).
13. Maes, M. et al. Acta Psychiat Scand 127, 344-354, (2013).
14. Clarke, G. et al. Mol Psychiatry 18, 666-673, (2013).
15. Cheung, S. G. et al. Front Psychiatry 10, 34, (2019).
16. Valles-Colomer, M. et al. Nat Microbiol 4, 623-632, (2019).
17. Ellul, P., et al, D. Front Neurol 9, 167, (2018).
18. Chen, Y. et al. Psychiatry Res 188, 224-230, (2011).
19. Saito, S et al., Am J Reprod Immunol 63, 601-610 (2010).
20. Meltzer-Brody, S. Dialogues Clin Neurosci 13, 89-100 (2011).
21. Mastorakos, G. & Ilias, I. Ann NY Acad Sci 997, 136-149, (2003).
22. Zhang, L., Zhang, J. & You, Z. Front Cell Neurosci 12, 306, (2018).
23. Rosenblat, J. D., et al Prog Neuropsychopharmacol Biol Psychiatry 53, 23-34, doi: 10.1016/j.pnpbp.2014.01.013 (2014).
24. Wohleb, E. S. et al; Psychoneuroendocrinology 37, 1491-1505, (2012).
25. Wohleb, E. S. et al. J Neurosci 31, 6277-6288, (2011).
26 Czeh, M., Gressens, P. & Kaindl, A. M. Dev Neurosci 33, 199-209, (2011).
27 Venneti, S., Lopresti, B. J. & Wiley, C. A. Glia 61, 10-23, (2013).
28 Yirmiya, R. et al. Adv Exp Med Biol 461, 283-316, (1999).
29 Rock, R. B. et al. Clin Microbiol Rev 17, 942-964, (2004).
30 Vollmer-Conna, U. et al. Psychol Med 34, 1289-1297, (2004).
31 Dowlati, Y. et al. Biol Psychiatry 67, 446-457, (2010).
32 Haapakoski, R., et al. Brain Behav Immun 49, 206-215, (2015).
33. Reichenberg, A. et al. Arch Gen Psychiatry 58, 445-452, (2001).
34. Grigoleit, J. S. et al. PLOS One 6, e28330, doi: 10.1371/journal.pone.0028330 (2011).
35. Harrison, N. A. et al., Biol Psychiatry 66, 407-414, (2009).
36. Yirmiya, R. Brain Res 711, 163-174, (1996).
37. Yirmiya, R. et al. Neuropsychopharmacol 24, 531-544, (2001).
38. Henry, C. J. et al. J Neuroinflammation 5, 15, (2008).
39. O'Connor, J. C. et al. Mol Psychiatry 14, 511-522, (2009).
40. O'Connor, J. C. et al. J Neurosci 29, 4200-209, (2009).
41. Dobos, N. et al., J Alzheimers Dis 28, 905-915, (2012).
42. Fenn, A. M. et al. Biol Psychiatry 76, 575-584, (2014).
43. Corona, A. W. et al. Brain Behav Immun 31, 134-142, (2013).
44. Li, Z. Q., et al., Biochem Biophys Res Commun 501, 933-940, (2018).
45. Garcia-Barrio, M., et al. EMBO J 19, 1887-1899, (2000).
46. Marton, M. J., et al. Mol Cell Biol 17, 4474-4489 (1997).
47. Paytubi, S., et al. Biochem J 409, 223-231, (2008).
48. Campbell, S. G., et al. J Cell Biol 170, 925-934, (2005).
49. Pestova, T. V. & Hellen, C. U. Nat Struct Mol Biol 13, 98-99, (2006).
50. Richard, M., et al. Genomics 53, 137-145, (1998).
51. Ota, M. et al. Immunogenetics 59, 45-52, (2007).
52. Zhu, H. et al. PLOS One 11, e0167212, (2016).
53. Arora, H. et al. Immunity 50, 418-431 e416, (2019).
54. Brunton, L., et al The Pharmacological Basis of Therapeutics. 12 edn, (McGraw Hill Professional, 2010).
55. Horowitz, M. A. et al. Int J Neuropsychopharmacol 18, (2014).
56. Abdel-Salam, O. M., et al Pharmacol Res 48, 157-165 (2003).
57. Roumestan, C. et al. Respir Res 8, 35, (2007).
58. Tynan, R. J. et al. Brain Behav Immun 26, 469-479, (2012).
59. Al-Amin, M. M., et al. Inflammopharmacology 21, 421-425, (2013).
60. Cattaneo, A. et al. Neuropsychopharmacology 38, 377-385, (2013).
61. Kenis, G. & Maes, M. Int J Neuropsychopharmacol 5, 401-412, (2002).
62. Kubera, M. et al. J Clin Psychopharmacol 21, 199-206 (2001).
63. Powell, T. R. et al. Eur Neuropsychopharmacol 23, 1105-1114, (2013).
64. Uher, R. et al., Am J Psychiatry 167, 555-564, (2010).
65. Malki, K. et al., Pharmacogenet Genomics 22, 765-776, (2012).
66. Powell, T. R. et al. J Psychopharmacol 27, 609-615, (2013).
67. Wilcox, S. M. et al. Plos One 12, e0175918, (2017).
68. Kimura-Ohba, S. et al. J Cereb Blood Flow Metab 36, 1731-1743, (2016).
69. Albert, P. R. J Psychiatry Neurosci 40, 219-221 (2015).
70. Yurist-Doutsch, S. et al., Plos One 11, e0159676, (2016).
71. Bartram, A. K et al. Appl Environ Microbiol 77, 3846-3852, (2011).
72. Schloss, P. D. et al. Appl Environ Microbiol 75, 7537-7541, (2009).
73. Hartmann, M. et al. ISME J 6, 2199-2218, (2012).
74. DeSantis, T. Z. et al. Appl Environ Microbiol 72, 5069-5072, (2006).
75. McMurdie, P. J. & Holmes, S. Plos One 8, e61217, (2013).
76. Christoffel, D. J. et al. J Neurosci 31, 314-321, (2011).
77. Coll, R. C. et al. Nat Med 21, 248-255, (2015).
78. Golden, S. A., et al Nat Protoc 6, 1183-1191, (2011).
79 McGirr, A., et al. Brain 140, 2210-2225, (2017).
80 Brady, A. M. & Floresco, S. B. Vis Exp, e52387 (2015).
81 Weiss, J. M., et al J Comp Physiol Psychol 72, 153-160 (1970).
82 Wagner, H. R., 2nd, Hall, T. L. & Cote, I. L. J Gen Psychol 96, 313-318, (1977).
83 Porsolt, R. D., et al., Arch Int Pharmacodyn Ther 229, 327-336 (1977).
84 Steru, L, et al., Psychopharmacology (Berl) 85, 367-370 (1985).
85 Liu, M. Y. et al. Nat Protoc 13, 1686-1698, (2018).
86 Sunyer, B., et al. Barnes maze, a useful task to assess spatial reference memory in the mice (2007).

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of monitoring an inflammatory or immune response, an inflammatory and/or autoimmune disease, said method comprising
a) collecting samples at pre-determined intervals from a subject;
b) measuring the level of ABCF1 in each of the samples collected from the patient; and
c) comparing the level of ABCF1 in each of the samples;
wherein a decrease in the level of ABCF1 over time indicates that the inflammatory or immune response is increasing and/or said disease is worsening and wherein an increase in the level of ABCF1 over time indicates that the inflammatory or immune response is decreasing and/or said disease is improving.

2. A method of diagnosing a disease or disorder, optionally associated with inflammatory and/or autoimmune diseases in a patient comprising the steps of:
a) collecting a sample from the patient;
b) measuring the level of ABCF1 in the sample collected from the patient; and
c) comparing the levels of ABCF1 with a predefined level of ABCF1;
wherein a correlation between the ABCF1 level in the patient sample and predefined ABCF1 levels indicates that the patient has said disease or disorder.

3. (canceled)

4. The method of claim 1, wherein said monitoring is for monitoring treatment of said disease,
wherein a decrease in the level of ABCF1 during a treatment indicates that the treatment is not effective and wherein an increase in the level of ABCF1 over time indicates the treatment is effective.

5. The method of claim 1, wherein the disease is an inflammatory and/or autoimmune disease is selected from arthritis, including but not limited to Rheumatoid Arthritis and other autoimmune arthritis, thyroid autoimmune diseases including but not limited to Grave's disease, cutaneous lupus erythematosus, autoimmune pancreatitis, inflammatory bowel disease including but not limited to Crohn's disease and ulcerative colitis and neuroinflammatory diseases and disorders including but not limited to Major Depressive Disorder (MDD) and multiple sclerosis (MS).

6. The method of claim 1, wherein the disease is Major Depressive Disorder.

7. The method of claim 1, wherein the disorder is addiction, addictive behaviour, compulsive disorder or inability to concentrate.

8. The method of claim 1, wherein the disease or disorder is post-COVID-19.

9. The method of claim 8, wherein the disorder is a neurological disorder post-COVID-19.

10. The method of claim 8, wherein the disorder is post-COVID-19 brain fog.

11. The method of claim 1, wherein ABCF1 and optionally other markers is measured using mass spectrometry or an immunoassay.

12. The method of claim 1, wherein circulating biomarker DNA or RNA is measured.

13. The method of claim 1, wherein the sample(s) is a cell sample.

14. The method of claim 13, wherein said cell sample(s) is a PBMC sample(s).

15. The method of claim 1, wherein the sample is a blood, plasma, serum, urine, sweat, cerebrospinal fluid, pleural fluid, bronchial lavages, sputum, peritoneal fluid, bladder washings, secretions, oral washings, swabs, isolated cells, tissue samples, touch preps, and fine-needle aspirates.

16. (canceled)
